SEQUENCE LISTING (6 sequences)

SEQ ID NO: 1 - DNA, 1476 bp, Artificial Sequence
  single chain fragment variable region nucleotide
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcgttaacaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaggagtaagaggagcaggctcctgcacagtgactacatgaacatgactccccgccgccccgggcccacccgcaagcattaccagccctatgccccaccacgcgacttcgcagcctatcgctccggtggcggatcccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 2 - DNA, 1476 bp, Artificial Sequence; identical to SEQ ID NO: 1
  single chain fragment variable region nucleotide

SEQ ID NO: 3 - DNA, 1479 bp, Artificial Sequence
  single chain fragment variable region nucleotide
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcgttaacaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgggtggcggatcccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 4 - protein, 533 aa, Artificial Sequence
  single chain fragment variable region peptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSVNTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELGGGSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 491 aa, Artificial Sequence
  single chain fragment variable region peptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSVNTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 492 aa, Artificial Sequence
  single chain fragment variable region peptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSVNTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0276012 A1
(43) Pub. Date: Sep. 4, 2025

(54) NOVEL CAR T-CELL PRODUCT AND METHOD OF PREPARATION THEREOF

(71) Applicant: Senthilkumar NATESAN, Ahmedabad (IN)

(72) Inventor: Senthilkumar NATESAN, Ahmedabad (IN)

(21) Appl. No.: 18/547,709

(22) PCT Filed: Feb. 25, 2022
(86) PCT No.: PCT/IB2022/051670

§ 371 Date: Aug. 24, 2023

(30) Foreign Application Priority Data
Feb. 25, 2021 (IN) 202121007954

(51) Int. Cl. A61K 35/17; A61K 40/11; A61K 40/31; A61K 40/42; C07K 14/705; C07K 14/725; C07K 16/28; C12N 5/0783; C12N 15/86

(52) U.S. Cl. CPC A61K 35/17; A61K 40/11; A61K 40/31; A61K 40/4211; C07K 14/7051; C07K 14/70517; C07K 14/70521; C07K 14/70578; C07K 16/2803; C12N 5/0636; C12N 15/86; A61K 2239/13; A61K 2239/17; A61K 2239/21; A61K 2239/23; C12N 2510/00; C12N 2740/15043

(57) ABSTRACT

The present invention provides engineering of T cells to express a Chimeric Antigen Receptor (CAR), and compositions comprising the same. The present invention related to novel CD19-targeting CAR T cell product comprising second and third generation CAR gene for treating B-cell disorders including leukaemia and lymphoma. The present invention is also related to process of preparing the novel CAR T cell products.

### FIELD OF THE INVENTION

The present invention is in the field of synthesis of novel CAR T cell product and method of its preparation. The present invention provides novel CD19-targeting CAR T cell product comprising second and third generation CAR gene for treating B-cell disorders including leukaemia and lymphoma.

### INCORPORATION BY REFERENCE OF SEQUENCE LISTING

The applicant hereby incorporates by reference the Sequence Listing filed concurrently herewith in electronic format as a text file named “Novel_CAR_T”; Date of creation May 31, 2024; Size of the file 19,883 bytes. The content of the Sequence Listing is hereby expressly incorporated herein by reference in its entirety.

The applicant asserts that the Sequence Listing, when combined with the present disclosure, fully describes and enables the invention claimed in the patent application.

### BACKGROUND OF THE INVENTION

CAR T-cell therapy is an immunotherapy which bolsters the body's immune system to fight against cancer. In CAR therapy, the T-cells (immune cells that attack foreign bodies) are extracted from the patient's blood and are genetically engineered in a lab to create a chimeric antigen receptor on the T-cell surface and then multiplied in large numbers. The engineered cells are then injected back into the patient's body, the CAR present on the T-cell binds directly to the antigens present on tumour cells. After binding of CAR with the antigen, the T-cell is activated which initiates killing of the tumour cells. CAR T-cell therapy is more effective than T-cell receptor (TCR) therapy as it can directly target the antigen present on a cell's surface. Unlike TCR therapy, chimeric antigen receptor doesn't require the protein to be processed or presented by major histocompatibility complex (MHC) for its recognition.

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering. CD19 is an attractive target for immunotherapy because the vast majority of B-acute lymphoblastic leukemia (B-ALL) uniformly express CD19, whereas expression is absent on non-hematopoietic cells as well as myeloid, erythroid and T cells and bone marrow stem cells. Clinical trials targeting CD19 on B-cell malignancies are underway with encouraging anti-tumor responses. Most infuse T cells genetically modified to express a chimeric antigen receptor (CAR) with specificity derived from the scFv region of a CD19-specific mouse monoclonal antibody FMC63. However, there is still a need to improve construction of CARs that show better compatibility with T-cell proliferation, in order to allow the cells expressing such CARs to reach significant clinical advantage.

U.S. Pat. No. 9,629,877B2 relates to CAR-expressing T-cells are producing using electroporation in conjunction with a transposon-based integration system to produce a population of CAR-expressing cells that require minimal ex vivo expansion or that can be directly administered to patients for disease (e.g., cancer) treatment.

EP3205720A1 relates to an object of the present invention is to provide CAR-expressing T cells that coexpress a chimeric antigen receptor (CAR) and a T cell immune function-enhancing factor and have a high immunity-inducing effect and antitumor activity, and to provide a CAR expression vector for the preparation of the CAR-expressing T cells.

CN108085342A relates to a preparation method of chimeric antigen receptor gene modified T lymphocyte, prepared CAR-T cell and application thereof. The preparation method comprises the following steps: S1, activating a CD4+ T cell and a CD8+ T cell which are obtained through separation; S2, infecting the CD4+ T cell and a CD8+ T cell which are processed in S1 with a recombinant lentivirus that carries EGFR-CAR, co-culturing an EGFR-CAR CD4+ T cell and an EGFR-CAR CD8+ T cell which are obtained after the infection treatment to promote proliferation of the EGFR-CAR CD8+ T cell.

CA2951355A1 relates to an ErbB2-specific NK-92 cell or cell line containing a lentiviral vector encoding a chimeric antigen receptor and preferably two vector integration loci in its cellular genome. It relates to the use of the ErbB2-specific NK-92 cell or cell line in the prevention and/or treatment of cancer, preferably ErbB2-expressing cancers.

CN105131126A relates to a chimeric antigen receptor for treating malignant tumor and a preparation method and application of the chimeric antigen receptor. The chimeric antigen receptor is an NY-ESO-1 specificity chimeric antigen receptor; specifically, the chimeric antigen receptor is protein formed by a variable region of an NY-ESO-1 TCR alpha chain, a variable region of an NY-ESO-1 TCR beta chain, a hinge region and a trans-membrane region of CD8, a CD28 intracellular signal structure region, a CD137 intracellular signal structure region and a CD3 zeta intracellular signal structure region in sequence from the amino terminal to the carboxyl terminal.

CA2945388A1 relates to a chimeric antigen receptors (CARs) and CAR-expressing T cells are provided that can specifically target cells that express an elevated level of a target antigen. It relates to a an isolated transgenic cell comprising an expressed chimeric T-cell receptor (CAR) targeted to an antigen, said CAR having a Kd of between about 5 nM and about 500 nM relative to the antigen.

The inventor of the present invention have identified a novel strategy to design and synthesize a switchable chimeric antigen receptor (CAR) involving the antigen binding domain of anti-CD19 antibody signalling domains of CD28, 41BB and CD3 molecules. The present inventor also describes the novel process of CAR T cell generation that could make the manufactured CAR T cells more safe and affordable.

### OBJECTIVE OF THE INVENTION

The main objective of the present invention is to provide a novel CAR T cell product of switchable second and third generation of chimeric antigen receptor (CAR) gene, where the antibody domain, costimulatory signalling domain, and CD3 molecule signalling domain can be easily switched or modified or changed.

Another objective of the present invention provides a novel chimeric antigen receptor (CAR) comprising third generation CD19-CD28-41BB-CD3z CAR gene by including specific restriction enzyme cut sites at the critical junction without compromising the functional integrity of CAR.

Another objective of the present invention provides a novel chimeric antigen receptor (CAR) comprising second generation CD190 specific restriction enzyme cut sites at the critical junction without compromising the functional integrity of CAR.

Another objective of the present invention provides a novel chimeric antigen receptor (CAR) comprising second generation CD19-41BB-CD3z CAR by including specific restriction enzyme cut sites at the critical junction without compromising the functional integrity of CAR.

Another objective of the present invention provides a novel chimeric antigen receptor (CAR) having restriction site at the junction between CD19 antibody region and hinge region of CD8a without losing functional integrity of the sequence.

Another objective of the present invention provides a novel chimeric antigen receptor (CAR) having restriction site at the junction between 3′ end of costimulatory domain and 5′ end of CD3 signalling domain.

Another objective of the present invention provides a novel antibody or signalling domain switchable chimeric antigen receptor (CAR) design by joining CD3, CD28, 41BB and antibody regions, where the antibody portion or the costimulatory signalling domain or the CD3 signalling domain can be switched by restriction digestion using the incorporated Hpa 1 and Bam HI and selected restriction enzyme that can cut the vector at sequence flanking the CAR gene.

Another objective of the present invention provides a novel process of manufacturing the CAR T product directly from the peripheral blood mononuclear cells (PBMC) without any need for purification of T cells, which makes the manufacturing cost effective and make the product affordable. It avoids the need for leukapheresis procedure for obtaining large number of blood T cells to generate CAR T cells.

Another objective of the present invention provides a novel process of manufacturing the CAR T product using the soluble anti-CD3 and anti-CD28 antibodies based activation of T cell without utilizing anti-CD3 bead or anti-CD28 bead, which makes the process cost effective and safer without any need for bead removal step.

Another objective of the present invention provides a novel process of manufacturing the CAR T product using T cells recovered from the in vitro cultures of macrophages, nurse macrophages, and lympho-myeloid niches following activation and/or addition of interleukin and infecting them with CAR gene containing lentiviral particles to generate CAR T cells.

Another objective of the present invention provides a novel process of transfecting 293T cells for generating lentiviral particles containing CAR gene using different transfection reagents for manufacturing CAR T cells.

Another objective of the present invention provides a novel process of introducing mutations deletions and insertions in the CAR gene.

Another objective of the present invention provides a novel process for estimating the functional efficacy of CAR T cells by measuring the depletion of CD19 positive cells during the co-culture of CAR T cells and target cells.

### SUMMARY OF THE INVENTION

The main aspect of the present invention is to provide a CAR T cell product comprising third generation chimeric antigen receptor gene CD19-CD28-41BB-CD3z comprising the nucleotide sequences of single chain variable region of anti-CD19 antibody, signalling domain of CD28, signalling domain of 41BB, transmembrane region CD8a, CD8aHinge region, and CD3z signalling domain along with leader and linker sequences, where in the CAR gene nucleotide sequence is as per SEQ ID No. 1.

The other main aspect of the present invention is to provide a CAR T cell product comprising second generation CAR gene CD19-CD28-CD3z comprising the nucleotide sequences of single chain variable region of anti-CD19 antibody, signalling domain of CD28, transmembrane region CD8a, CD8a Hinge region, and CD3z signalling domain along with leader and linker sequences, where in the CAR gene nucleotide sequence is as per SEQ ID No. 2.

Another main aspect of the present invention is to provide a CAR T cell product comprising second generation CAR gene CD19-41BB-CD3z comprising the nucleotide sequences of single chain variable region of anti-CD19 antibody, signalling domain of 41BB, transmembrane region CD8a, CD8aHinge region, and CD3z signalling domain along with leader and linker sequences, where in the CAR gene nucleotide sequence is as per SEQ ID No. 3.

Another aspect of the present invention is to provide a CAR T cell product comprising an engineered cell comprising DNA encoding a chimeric antigen receptor (CAR) protein comprises an amino acid sequence which is at least 90% identical to SEQ ID No. 4 or 5 or 6.

Another aspect of the present invention is to provide a process of cloning of CD19 CAR sequence in the Lentiviral vector comprises steps of,
a) Cloning of purified CAR gene product into a cloning vector, used to transform the competent cells,
b) Spreading the transformed competent cells into a LB (Luria broth) agar plate containing ampicillin,
c) Incubating the plates in an incubator at 37° C. overnight,
d) Growing the large number of colonies in the agar plate in the LB broth,
e) Screening of the clones using polymerase chain reaction and identifying the clones positive for the desired insert,
f) Purifying the plasmids from the clones of interest,
g) Analysing the sequence of CAR clones by sequencing shows presence of mutations either in the form of deletion or insertions wherein the desired clone was selected for further cloning in to the lentiviral vector.
h) For cloning the CAR gene into the lentiviral vector, a double digest of cloning vector and the lentiviral vector wherein loading them on to agarose gel and purifying them from the gel,
i) Setting up ligation reaction using the linearized lentiviral vector and the CAR gene in the presence of T4 ligase,
j) Transforming the competent cells using the ligation mixture by spreading over the LB agar plate and keeping in an incubator overnight,
k) Growing the clones observed on the plate in LB broth medium,
l) Confirming the presence of gene of interest was by polymerase chain reaction and confirming that all the clones had insert of the expected size and selecting the clone having intact reading frame and no mutation.

Another aspect of the present invention is to provide a process of preparation of lenti-CD19 CAR viral vector comprises the steps of,
a) Confirming the sequence of all the three clones, one with both CD28 and 41BB, and other two single domain clones (only CD28 and only 41BB),
b) Preparing the Midiprep of all the clones and used that for transfection using 293T cells, Preparing the maxiprep of the lentiviral transfer vectors containing CD19-CAR and packaging vectors using the Endotoxin free Maxiprep plasmid preparation kit, for this purpose, growing the transformed bacteria harbouring the clone in 700 ml of LB broth and pelleting the cells by centrifuging the both culture at 4000 rpm for 15 minutes, after centrifugation, suspending the cell pellet in the resuspension buffer by vortexing and the plasmid extraction, dissolving the DNA pellet in 1.2 ml of TE buffer,
c) Observing the concentration of plasmids in Midiprep was much lower compared to the Maxiprep as expected, then several aliquots of each plasmid can be made and stored at −20° C. until further use.

Another aspect of the present invention is to provide a process of transfecting 293T cells for generating lentiviral particles containing CAR gene comprises the steps of,
a) Using 293 T cell line for transfection for preparing lentiviral particles,
b) Culturing the 293 T cells in RPMI medium containing 10% fetal bovine serum and L-glutamine without antibiotics,
c) Using the purified plasmids for the transfection of 293T cells
d) Transfecting the purified lentiviral transfer vector containing CAR gene along with the packaging vectors into 293T cells.
e) One the day prior to the transfection, seeding the 293T cells on to a T25 culture flask in 5 ml of RPMI medium with 10% FBS without antibiotics, the flask contains monolayer confluence of 60-70% next day and it is used for transfection,
f) Diluting the plasmids in 0.5 ml of Opti-MEM medium in tube-1 and the transfection reagent was diluted in another 0.5 ml of Opti-MEM medium in tube-2,
g) Allowing the plasmids to stand for 30 minutes at room temperature, then, both the plasmid (tube-1) and transfection reagent (tube-2) content were mixed together and allowed to stand for 45 minutes,
h) Taking out the 293T cell flask from the incubator and removing the old media and discarding, washing the monolayer with 2 ml of Opti-MEM medium gently, and adding fresh 1 ml of Opti-MEM,
i) Adding the plasmid-transfection reagent complex to the flask gently and mixing well gently for uniform distribution of the complex,
j) Incubating the flask at 37° C. incubator with 5% CO2,
k) Adding 2 ml of fresh Opti-MEM medium after 8 hours of incubation and keeping back the flask in to the CO2 incubator for overnight incubation,
l) Adding 4 ml of RPMI medium containing 20% FBS without antibiotics on next day and incubating back in to the CO2 incubator and observing the cells everyday under the inverted microscope
m) Harvesting the supernatant containing viral particles after 72 hours following transfection, and determining the concentration of viral particles by measuring the p24 protein using standard commercially available kit,
n) Aliquoting and storing the lentiviral particles at minus 80° C. freezer until further use.

### BRIEF DESCRIPTION OF THE DRAWING

FIG. 1. Generation of three lentiviral vectors containing CD19 targeting CAR gene.

FIG. 2. Screening of the clones for the presence of desired third generation CAR gene insert by PCR. A total of 128 clones screened and only few had insert of expected size.

FIG. 3. Confirmation of the selected clones for the presence of gene of interest by polymerase chain reaction. All the clones were found positive for the insert of expected size.

FIG. 4. Generation of chimeric antigen receptor gene with only one costimulatory signalling domain using inverse PCR. Lane 1-3 shows the amplification with only CD28 domain and the lane 4-6 shows the amplification with only 41BB domain. The three lanes of each set indicate different concentration of template DNA (1, 2, 4 μl) used for amplification. P is the positive control with both domains which shows the size slightly higher than other products due to the presence of two domains. N is negative control.

FIG. 5. Confirmation of the presence of gene of insert in selected clones. Lane 1-9 indicate the presence/absence of insert in clone 1-9. P is the positive control, M is the 100 bp ladder marker, and the N is the negative control.

FIG. 6. Confirmation of the presence of gene of insert in selected clones. Lane 1-12 indicate the clone 1-12. P is the positive control, M is the 100 bp ladder marker, and the N is the negative control.

FIG. 7. Gel image showing presence of amplification of expected size in the clone-1 and absent in the clone-2. As the positive control is the double domain clone, its product size is slightly higher than the single domain clone.

FIG. 8. Gel image showing presence of amplification of expected size in the clone-1 and clone-2. As the positive control is the double domain clone, its product size is slightly higher than the single domain clones.

FIG. 9. Confirmation of the three different CAR genes with single and double signalling domain in three different lentiviral plasmids. Lane 1-double signalling domain containing third generation CAR gene and Lane 2 and 3 with single signalling domain containing second generation CAR gene, where lane 4 is negative control.

FIG. 10. Gel image showing the concentration of the large scale preparations of three lentiviral plasmids. Marker is 100 bp ladder.

FIG. 11. Shows the morphology and confluence of 293T cells on day 3 following transfection.

FIG. 12. Transfection of 293T cells with two transfection reagents with different reporter gene to assess the expression. Both induced high level expression of the gene, however, the SN01 induced expression was slightly higher than the SN02.

FIG. 13: Image shows the expression of CAR on 293T cells on day 3 following transfection. The cells were fixed in 4% PFA and stained using anti-Fab FITC conjugate and imaged under a fluorescent microscope.

FIG. 14. Image shows the detection of the CAR gene expression in 293T cells by flow cytometry. The cells were transfected with lentiviral vector carrying CAR gene and harvested on day 4. The plots 1 and 2 from left were from the unstained tube whereas the plots 2 and 3 were from the tube stained with anti-Fab-FITC antibody and then acquired in a flow cytometer, which indicated 86% of cells expression CAR on the surface.

FIG. 15. The T cells 2 days following the anti-CD3 and CD28 activation prior to the infection showed active proliferation and expansion in the culture vessels.

FIG. 16. Image shows the characterization of the T cells based on the CD3 and CD4 surface markers by flow cytometry using the activated T cells from two different donors.

FIG. 17. The T cells 3 days following the anti-CD3 and CD28 activation prior to the infection which showed active proliferation and expansion of cells with large proliferating clumps.

FIG. 18. Shows the individual antibody stained T cells transduced with Lenti-CAR-19 viral particles on day 8 following infection. The cells shown in the first top row is unstained for any antibody, the cells in second row is stained only for Fab antibody, cells in the third row is stained only for CD3 antibody, and the cells in the fourth row is stained only for CD4 antibody.

FIG. 19. Shows the characterization of the T cells by staining with anti-Fab-FITC (A-mock infected, B [flask-1] and C [flask2]-LV infected day 8 post infection; D-day 3 post infection) following infection with mock transfected or Lentiviral transfected 293T cells supernatant. The cells were stained in separate tubes and acquired in the flow cytometer and analysed using the Flowing software. Analysis showed the expression of CAR molecules on the T cells.

FIG. 20. T cells transduced with FITC labelled CAR bind antibody for the expression of CAR observed by analysing them under a fluorescent microscope.

FIG. 21. Image showing the detection of the CAR gene in the T cells. The testing was done by two PCR approaches, one detecting the whole gene and another detecting the partial gene. The cells were tested in duplicate flasks (F1 and F2).

FIG. 22. Engineered K562-19 cells along with the parental K562 is shown at different magnification.

FIG. 23. The expression of CD19 on the surface of engineered K562 cells was confirmed by staining the cells with anti-CD19-APC antibody and analysing them in the flow cytometer. The expression of CD19 on the surface of K562 cells is shown in the Figure.

FIG. 24. Histogram shows the expression of CD19 in K562 cells. When these K562 and K562-19 cells were cocultured with CD19 specific CAR T cells, killing of only K562-19 cells was observed, leading to the media pH very alkaline due to lack of proliferating K562-19 cells in the coculture and the normal K562 without expressing CD19 were not killed and then able to proliferate well in the presence of CD19 specific CAR T cells, lead to the pH of the media very acidic.

FIG. 25. Image shows the coculture of control T cells (CONT) and CD19 specific CAR T cells (dmCART) with K562 cells expressing CD19 (K562-19). The T cells and 562-19 cells were mixed at 10:1 ratio.

FIG. 26. Bar diagram shows the spheroid numbers quantified in each field under bright field microscopy for each coculture condition. K562.19-K562 cells expressing CD19, CART-T cells expressing CD19 specific CAR, and CONT-T cells not-expressing CAR.

FIG. 27. Image shows the coculture of control T cells (CONT) and CD19 specific CAR T cells (CART) with K562 cells expressing CD19 (K562-19) and control K562 cells without expressing CD19. The T cells and 562 cells were mixed at 10:1 ratio.

FIG. 28. Bar diagram shows the spheroid numbers quantified in each field under bright field microscopy at 40× magnification for each coculture condition. K562-K562 cells not expressing CD19, K562.19-K562 cells expressing CD19, CART-T cells expressing CD19 specific CAR, and CONT-T cells not-expressing CAR.

FIG. 29. Total number of cells at different intervals following co-culture of different CAR-T cells and control T cells with K562 and K562-19 cells.

FIG. 30A. CAR T cells cocultured with K562-19 cells at 1:5 ratio. Cells were analysed on day2 following coculture. Left column of the dot blot is control T cells and the three columns in the middle are different CAR T cells cocultured with K562-19 cells. The last column is K562-19 only control cells.

FIG. 30B. CAR T cells cocultured with K562-19 cells at 1:5 ratio. Cells were analysed on day 4 following coculture. Left column of the dot blot is control T cells and the three columns in the middle are different CAR T cells cocultured with K562-19 cells. The last column is K562-19 only control cells.

FIG. 31. Co-cultured cells stained with propidium iodide and analysed in the flow cytometer. The dot blot and histogram in the left is control T cells and the other three columns in the right are CAR T cells cocultured with K562-19 cells.

FIG. 32. Dose dependent cytotoxicity of T cells killing cancer cells. The histogram rows 1, 2 and 3 indicate the cancer cells and T cells coculture ratio of 1:2, 1:5, and 1:10. Left column of the dot blot is control T cells and the three columns in the middle are different CAR T cells cocultured with K562-19 cells. The last column is K562-19 only control cells.

FIG. 33. Dose dependent cytotoxicity of T cells killing cancer cells at 1:5 ratio of coculture. The dot blot and histogram in the Left column is control T cells and the three other columns are different CAR T cells cocultured with K562-19 cells.

FIG. 34. Dose dependent cytotoxicity of T cells killing cancer cells at 1:5 ratio of coculture analysed up to day 6 in culture.

FIG. 35. Dose dependent cytotoxicity of T cells killing cancer cells at 1:10 ratio of coculture. The dot blot and histogram in the Left column is control T cells and the three other columns are different CAR T cells cocultured with K562-19 cells.

FIG. 36. Dose dependent cytotoxicity of T cells killing cancer cells at 1:10 ratio of coculture analysed up to day 6 in culture.

FIG. 37. Cytotoxicity of T cells on cancer cells at 1:5 ratio of coculture and complete elimination of cancer cells from the cultures. The dot blot and histogram in the Left column is control T cells and the three other columns in the middle are different CAR T cells cocultured with K562-19 cells. The last column in the right is K562-19 only control cells. The bar diagram in the right left and right shows the percentage reduction in the total K562-19 cell and CD19+ve K562-19 cells. The bar diagram in the middle shows the percentage reduction of K562-19 compared to the control during the K562-19 cell and CD19 specific CAR T cell co-culture. Three different CAR T cells (1328 having both CD28 signalling domain, 8T021 having 41BB signalling domain, 1526 having both CD28 and 41BB signalling domain) were used for co-culture. All three newly developed CD19 specific CAR T cells showed 90% and above reduction of the K562-19 cells compared to control T cells.

FIG. 38. Characterization of the blood obtained from the relapsed B-ALL patients. Only a small fraction on the cells in lymphocyte gates are T cells.

FIG. 39. The activated T cells collected on day 2 and day 3 in culture and stained with different antibodies to characterize the presence of different cells types.

FIG. 40. CAR T cells cocultured with K562-19 cells at 1:10 ratio. Cells were analysed on day 3 following coculture using the flow cytometer after staining with propidium iodide. The last column shows gated K562-19 cells for propidium staining.

FIG. 41. CAR T cells cocultured with K562-19 cells at 1:5 ratio. Cells were analysed on day 4 following coculture. Top row of the dot blot/histogram is control T cells and the middle and lower row are different CAR T cells cocultured with K562-19 cells. The second column is shown the analysis of CD19+of K562-19 cells without any gate and the third column three shows the analysis of cells with gating and the fourth column shows the presence of T cells stained with CD3 antibody.

FIG. 42. Cytotoxicity of T cells on cancer cells at 1:10 ratio of coculture and elimination of cancer cells from the cultures. The dot blot and histogram in the top row is control T cells and the two rows in the middle are different CD19 specific CAR T cells cocultured with K562-19 cells. The bar diagram in the right left and right shows the percentage of the total K562-19 cell and CD19+ve K562-19 cells. The bar diagram in the middle shows the percentage reduction of K562-19 compared to the control in the cell co-culture. Two different CAR T cells (8T021 having 41BB signalling domain with two different transfection protocol) were used for co-culture. The ALL patient derived CD19 specific CAR T cells showed 82.6% reduction of the K562-19 cells compared to control T cells.

### DETAILED DESCRIPTION OF THE INVENTION

Unless defined otherwise, all technical and scientific terms used herein have the same meaning as commonly understood by one of ordinary skill in the art to which the invention pertains. The term “a” and “an” refers to one or to more than one (i.e., to at least one) of the grammatical object of the article. The term “about” when referring to a measurable value such as an amount, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signalling domain (also referred to herein as “an intracellular signalling domain”) comprising a functional signalling domain derived from a stimulatory molecule and/or costimulatory molecule.

The term “signalling domain” refers to the functional portion of a protein which acts by transmitting information within the cell to regulate cellular activity via defined signalling pathways by generating second messengers or functioning as effectors by responding to such messengers.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV and FIV are all examples of lentiviruses.

The term “lentiviral vector” refers to a vector derived from at least a portion of a lentivirus genome. Examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “parenteral” administration of an immunogenic composition includes, e.g., subcutaneous (s.c), intravenous (i.v.), intramuscular (i.m.), or intrasternal injection, intratumoral, or infusion techniques.

The present invention relates to novel designing and synthesis of anti-CD19 CAR gene.

The main embodiment of the present invention is to provide a novel CAR T cell product of switchable second and third generation of chimeric antigen receptor (CAR) gene, where the antibody domain, costimulatory signalling domain, and CD3 molecule signalling domain can be easily switched or modified or changed.

Another embodiment of the present invention provides a novel chimeric antigen receptor (CAR) involving third generation chimeric antigen receptor gene CD19-CD28-41BB-CD3z comprising the nucleotide sequences of single chain variable region of anti-CD19 antibody, signalling domain of CD28, signalling domain of 41BB, transmembrane region CD8a, CD8aHinge region, and CD3z signalling domain along with leader and linker sequences, where in the CAR gene nucleotide sequence is as per SEQ ID No. 1.

Another preferred embodiment of the present invention provides a novel chimeric antigen receptor (CAR), second generation CAR gene CD19-CD28-CD3z comprising the nucleotide sequences of single chain variable region of anti-CD19antibody, signalling domain of CD28, transmembrane region CD8a, CD8a Hinge region, and CD3z signalling domain along with leader and linker sequences, where in the CAR gene nucleotide sequence is as per SEQ ID No. 2.

Another preferred embodiment of the present invention provides a second generation CAR gene CD19-41BB-CD3z comprising the nucleotide sequences of single chain variable region of anti-CD19 antibody, signalling domain of 41BB, transmembrane region CD8a, CD8aHinge region, and CD3z signalling domain along with leader and linker sequences, where in the CAR gene nucleotide sequence is as per SEQ ID No. 3.

As per one embodiment, the third generation CD19-CD28-41BB-CD3z CAR gene sequence comprising DNA encoding a chimeric antigen receptor (CAR) protein which is at least 90% identical to SEQ ID No: 1 and the second generation CAR gene sequence selected from CD19-CD28-CD3z and CD19-41BB-CD3z sequences comprising DNA encoding a chimeric antigen receptor (CAR) protein which is at least 90% identical to SEQ ID No: 2 and 3, respectively.

As per one embodiment, the CAR gene sequence is cloned into a DNA expression vector, lentiviral vector, adenoviral vector, adeno-associated viral vector, and transposon vector.

As per one embodiment, the CAR T cell product comprises an engineered cell comprising DNA encoding a chimeric antigen receptor (CAR) protein comprises an amino acid sequence which is at least 90% identical to SEQ ID No: 4 or 5 or 6, wherein DNA encoding the CAR is integrated into the genome of the cell or remain as an extra chromosomal DNA.

As per another embodiment, the CARs are designed in a modular fashion that typically consists of an extracellular target-binding domain, a hinge region, a transmembrane domain that anchors the CAR to the cell membrane, and one or more intracellular domains that transmit activation signals. Depending on the number of costimulatory domains, CARs can be classified into first (CD3z only), second (one costimulatory domain+CD3z), or third generation CARs (more than one costimulatory domain+CD3z). Introduction of CAR molecules into a T cell successfully redirects the T cell with additional antigen specificity and provides the necessary signals to drive full T cell activation.

As per another embodiment, the Leader sequence is the first part which causes CAR protein expression on T cell membrane after secretion from Golgi complex.

Another embodiment of the present invention provides a novel chimeric antigen receptor (CAR) having restriction site at the junction between CD19 antibody region and hinge region of CD8a without losing functional integrity of the sequence.

As per another embodiment, the hinge, also referred to as a spacer, is the extracellular structural region of the CAR that separates the binding units from the transmembrane domain.

Another embodiment of the present invention provides a novel chimeric antigen receptor (CAR) having restriction site at the junction between 3′ end of costimulatory domain and 5′ end of CD3 signalling domain. This allows switching of costimulatory domain with as required without altering the antibody sequence or CD3 signalling domain.

As per one embodiment, the novel CAR T cell product allows replacing the antibody domain or the signalling domain with an alternative antibody domain or alternative signalling domain without affecting the functional integrity of the vector by restriction digestion.

As per one embodiment, the alternative antibody domain are selected from B7-H3, BCMA, CAIX, CD123, CD133, CD138, CD147, CD19, CD22, CD30, CD33, CD38, CD4, CD5, CD7, CD70, CEA, EGFR, EGFRVIII, FAP, EpCAM, FOLR1, GPC3, Her2, Her3, HGFR, IL13RA2, MSLN, MUC1, MUC16, Nectin-4, NKG2D, PSCA, PSMA, ROR1, SLAMF7, uPAR and VGFR2 antibody sequences.

As per one embodiment, the alternative signaling domain are selected from ICOS, OX40 and FcεR1-γ.

As per another embodiment, the restriction enzyme recognizes and bind to specific sequences of DNA, called restriction sites. Each restriction enzyme recognizes just one or a few restriction sites. When it finds its target sequence, a restriction enzyme will make a double-stranded cut in the DNA molecule.

Another embodiment of the present invention provides a novel antibody or signalling domain switchable chimeric antigen receptor (CAR) design by joining CD3, CD28, 41BB and antibody regions, where the antibody portion or the costimulatory signalling domain can be switched by restriction digestion using the incorporated Hpa 1 and Bam HI and selected restriction enzyme that can cut the vector at sequence flanking the CAR gene.

As per another embodiment, the Hpa I is a restriction endonuclease that is used to cleave DNA at the recognition site 5′-GTT/AAC-3′, generating fragments with blunt ends.

As per another embodiment, the Bam HI is a type II restriction endonuclease, having the capacity for recognizing short sequences of DNA and specifically cleaving them at a target site.

As per one embodiment of the present invention, a novel process of manufacturing the CAR T product directly from the peripheral blood mononuclear cells (PBMC) without any need for purification of T cells, where the T cells are activated using soluble anti-CD3 and anti-CD28 antibodies and addition of interleukin, and then infecting using CAR gene containing lentiviral particles to generate CAR T cells. This make the manufacturing cost effective and make the product affordable. It avoids the need for leukapheresis procedure for obtaining large number of blood T cells to generate CAR T cells. The Leukapheresis is an expensive laboratory procedure in which white blood cells are separated from a sample of blood. It is a specific type of apheresis, the more general term for separating out one particular constituent of blood and returning the remainder to the circulation.

As per one embodiment of the present invention, a novel process of manufacturing the CAR T product using T cells recovered from the in vitro cultures of macrophages, nurse macrophages, and lympho-myeloid niches following activation and/or addition of interleukin and infecting them with CAR gene containing lentiviral particles to generate CAR T cells.

As per one embodiment, the interleukin used in the manufacturing of CAR T cell product are selected from interleukin-2, interleukin-15, interleukin-12, interleukin-18, interleukin-21 and interleukin-7.

As per another embodiment the present invention, a novel process of manufacturing the CAR T product using the soluble anti-CD3 and anti-CD28 antibodies based activation of T cell without utilizing anti-CD3 bead or anti-CD28 bead, which makes the process cost effective and safer without any need for bead removal step.

Another embodiment of the present invention provides a novel process of designing the CAR T having novel process of transducing the T cells with lentiviral particles.

As per one embodiment, the pharmaceutical composition comprising the CAR T cell product with a pharmaceutically acceptable carrier, diluent or excipient, and optionally one or more further pharmaceutically active polypeptides and/or compounds.

As per one embodiment, the novel CAR T cell product is used for parenteral or local administration.

Another embodiment of the present invention provides a novel process of introducing mutations deletions and insertions in the CAR gene by designing specific primers and applying novel PCR approach.

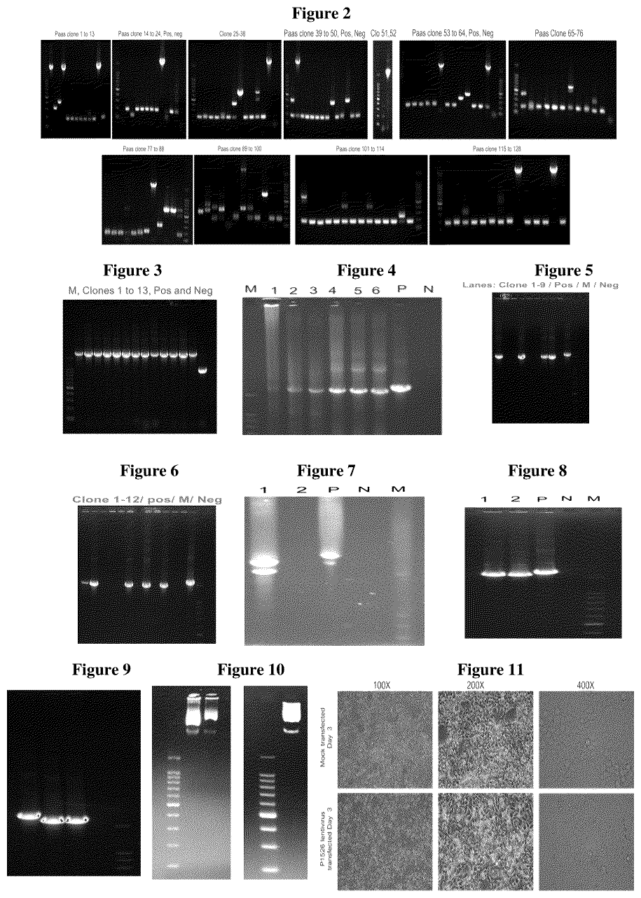

As per another embodiment the novel process of Cloning of CD19 CAR sequence in the Lentiviral vector comprises of:
a) cloning of purified CAR gene product into a cloning vector, used to transform the competent cells,
b) spreading the transformed competent cells into a LB (Luria broth) agar plate containing ampicillin,
c) incubating the plates in an incubator at 37° C. overnight,
d) growing the large number of colonies in the agar plate in the LB broth,
e) screening of the clones using polymerase chain reaction and identifying the clones positive for the desired insert,
f) purifying the plasmids from the clones of interest,
g) analysing the sequence of CAR clones by sequencing shows presence of mutations either in the form of deletion or insertions wherein the desired clone was selected for further cloning in to the lentiviral vector.
h) for cloning the CAR gene into the lentiviral vector, a double digest of cloning vector and the lentiviral vector wherein loading them on to agarose gel and purifying them from the gel,
i) setting up ligation reaction using the linearized lentiviral vector and the CAR gene in the presence of T4 ligase,
j) transforming the competent cells using the ligation mixture by spreading over the LB agar plate and keeping in an incubator overnight,
k) growing the clones observed on the plate in LB broth medium,
l) confirming the presence of gene of interest was by polymerase chain reaction and confirming that all the clones had insert of the expected size and the direction of insertion was as expected as represented in FIG. 3 and selecting the clone having the presence of intact reading frame and no mutation.

As per another embodiment the process of generating chimeric antigen receptor with only CD28 or 41BB signalling domain comprises of:
a) performing the per with the set of primers with overlapping sequences containing the deletion of one of the costimulatory domain,
b) performing the second round using another set of primers to amplify the full length product with desired deletion of one costimulatory domain,
c) purifying the amplified product from the agarose gel and cloning it the cloning plasmid,
d) carrying out the ligation of the purified product and cloning vector and transforming the competent cells with the ligation mix,
e) spreading the cells on the lb agar plate and keeping in the incubator overnight,
f) picking and growing the cells on lb medium and selecting the clones for the presence of insert by polymerase chain reaction.

As per another embodiment of the present invention, the process of generating chimeric antigen receptor with only 41BB signalling domain comprises, purifying the amplification product with only 41BB alone and ligating with cloning vector and transformed into competent cells by spreading on to the LB agar plate and keeping in the incubator. The gel image showing result of first 12 clones is represented in FIG. 6. Selecting the clones which are found positive for the insert and of expected size, cloning the single signalling domain containing second generation CAR gene into the lentiviral vector, purifying the fragment from the double digested vector and ligated with the linearized lentiviral vector, transforming the bacteria with the ligation mixture and spreading on to the LB agar plate, testing the clones for the presence of insert.

As per another embodiment of the present invention, the process of generating chimeric antigen receptor with CD28 and 41BB signalling domain comprises, screening two colonies using two primer sets in PCR for the insert of expected size as represented in FIG. 7, screened the two colonies from the 41BB domain by PCR for the presence of desired product as represented in FIG. 8, sequencing the CD28domain clone and 41BB domain clone and confirming the presence of intact sequence in correct reading frame.

As per another embodiment of the present invention, preparation of lenti-CD19 CAR viral vector and virus particles comprises, confirming all the three CAR containing plasmids again by using polymerase chain reaction as represented in FIG. 9, Large scale preparation of plasmids and checking it's concentration in the agarose gel as represented in FIG. 10, using these for transfecting the 293T cells and making the viral particles.

As per another embodiment of the present invention, preparation of lenti-CD19 CAR viral vector comprises
a) confirming the sequence of all the three clones, one with both CD28 and 41BB, and other two single domain clones (only CD28 and only 41BB).
b) preparing the midiprep of all the clones and used that for transfection using 293T cells, preparing the maxiprep of the lentiviral transfer vectors containing CD19-CAR and packaging vectors using the endotoxin free maxiprep plasmid preparation kit, for this purpose, growing the transformed bacteria harbouring the clone in 700 ml of LB broth and pelleting the cells by centrifuging the both culture at 4000 rpm for 15 minutes, after centrifugation, suspending the cell pellet in the resuspension buffer by vortexing and the plasmid extraction, dissolving the DNA pellet in 1.2ml of TE buffer.
c) Observing the concentration of plasmids in Midiprep was much lower compared to the Maxiprep as expected, then several aliquots of each plasmid can be made and stored at −20° C. until further use.

Another embodiment of the present invention provides a novel process of transfecting 293T cells for generating lentiviral particles containing CAR gene using different transfection reagents for manufacturing CAR T cells.

As per another embodiment the present invention provides a novel CAR T cell product which is used for treating B-cell disorders including leukaemia, lymphoma, in pediatric B-ALL, CLL, and NHL.

As per another preferred embodiment of the present invention, a novel process of transfecting 293T cells for generating lentiviral particles containing CAR gene comprises
a) using 293 T cell line for transfection for preparing lentiviral particles,
b) culturing the 293 T cells in RPMI medium containing 10% fetal bovine serum and L-glutamine without antibiotics,
c) using the purified plasmids for the transfection of 293T cells and transfecting the purified lenti-CD19 CAR transfer vector along with the packaging vectors into 293T cells.
d) one the day prior to the transfection, seeding the 293T cells on to a T25 culture flask in 5 ml of RPMI medium with 10% FBS without antibiotics, the flask contains monolayer confluence of 60-70% the next day and it is used for transfection,
e) diluting the plasmids in 0.5 ml of Opti-MEM medium in tube-1 and the transfection reagent was diluted in another 0.5 ml of Opti-MEM medium in tube-2,
f) allowing the plasmids to stand for 30 minutes at room temperature, then, both the plasmid (tube-1) and transfection reagent (tube-2) content were mixed together and allowed to stand for 45 minutes,
g) taking out the 293T cell flask from the incubator and removing the old media and discarding, washing the monolayer with 2ml of Opti-MEM medium gently, and adding fresh 1 ml of Opti-MEM,
h) adding the plasmid-transfection reagent complex to the flask gently and mixing well gently for uniform distribution of the complex and incubating the flask at 37° C. incubator,
i) adding 2 ml of fresh Opti-MEM medium after 8 hours of incubation and keeping back the flask in to the CO2 incubator for overnight incubation,
j) adding 4 ml of RPMI medium containing 20% FBS on next day and incubating back in to the CO2 incubator and observing the cells everyday under the inverted microscope as represented in FIG. 14,
k) harvesting the supernatant containing viral particles after 72 hours following transfection, and determining the concentration of viral particles by measuring the p24 protein using standard commercially available kit,
l) aliquoting and storing the lentiviral particles at minus 80° C. freezer until further use.

The inventors found that the viral particle concentration was 5-6 fold lower in the transfection using Midiprep plasmids compared to Maxiprep plasmids. These lenti-CD19 CAR viral particles were used to transduce primary human activated T cells to generate CD19 targeting CAR T cells.

Another embodiment of the present invention provides a novel process of detecting the CAR gene persistency by PCR by amplifying the partial CAR gene or full length CAR gene using different primers during the manufacturing of CAR T cells or to understand the persistence of CAR T cells in the body upon administration. Another embodiment of the present invention provides a novel process for estimating the functional efficacy of CAR T cells by measuring the depletion of CD19 positive cells during the co-culture of CAR T cells and target cells.

As per one embodiment of the present invention, a novel process of generation of CD4positive T cells using the T cells obtained from the Nurse macrophage cultures or from the T cells obtained from Lympho-myeloid niches or from the T cells obtained from CD3negativeCD14negative fraction of overnight PBMC cultures.

As per another embodiment the present invention provides advantages as compared to the prior art:
1) Novel switchable CAR design by joining CD3, CD28, 41BB and antibody regions, where the antibody portion or the signalling domains can be switched by restriction digestion using specific restriction enzymes.
2) Avoid the need for leukapheresis procedure for obtaining T cells to generate CAR T cells.
3) Generation of CAR T cells using cells of peripheral blood mononuclear cells without any need for purification of T cells or from T cells recovered from the Nurse macrophage cultures or from the T cells obtained from Lympho-myeloid niches or from the T cells obtained from CD3negativeCD14negative fraction of overnight PBMC cultures.
4) Generation of CD4positive CAR T cells using the T cells recovered from the Nurse macrophage cultures or using the T cells obtained from Lympho-myeloid niches or from the T cells obtained from CD3negativeCD14negative fraction of overnight PBMC cultures.
5) Activation of the T cells using soluble anti-CD3 and anti-CD28 antibodies so no hazard of using beads and no need for bead separation step.
6) Generation of lentiviral particles in the same culture medium used for growing T cells where the 293T cells are adapted to grow in RPMI medium and the same medium is used for growing the T cells to improve the biocompatibility of lentiviral particles during transduction.
7) Transduction of the T cells using the lentiviral particles in small volume and then gradual increase of culture media volume.
8) Safer and Cost effective approach for manufacturing CAR T cells and increases the success of generating CAR T cells from cancer patients.

The following examples are illustrated to describe the scope of the invention.

### EXAMPLE-1: PREPARATION OF LENTI-CD19 CAR VIRAL PARTICLES

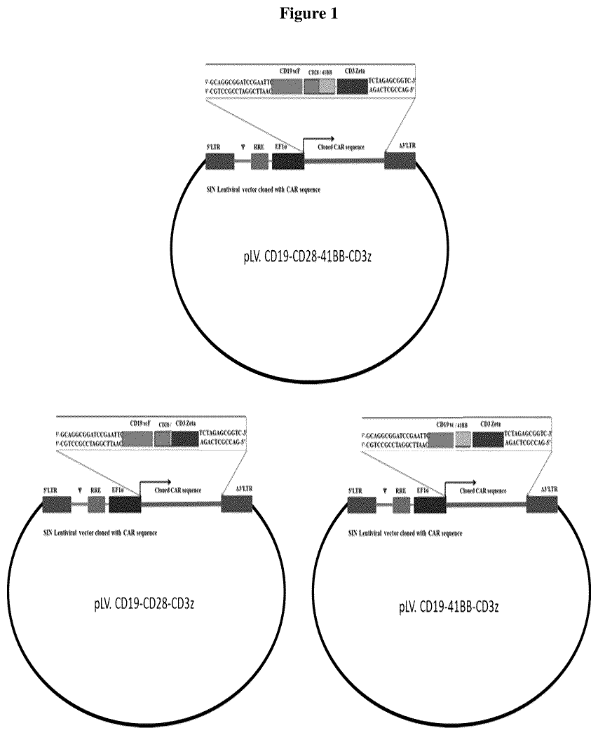

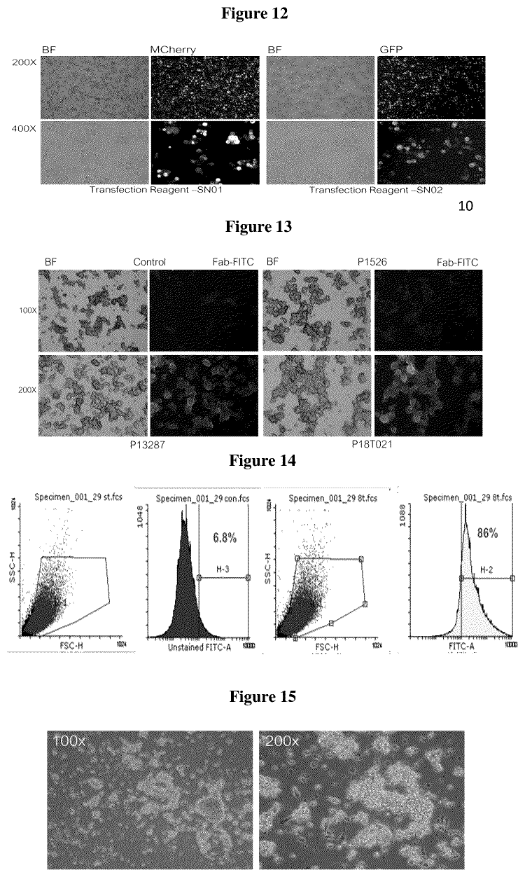

The CD19 targeting CAR gene was designed by introducing the restriction enzyme Hpa I cut site at the junction of ScFV of FMC63 antibody and the downstream sequence including hinge, transmembrane, costimulatory signalling domains. Similarly, the restriction enzyme Bam HI cut site was included at the junction of the Costimulatory domain and CD3z signalling domain. This novel design makes the CAR gene to be easily manipulated by replacing specific regions of interest. The gene was synthesised and cloned into a cloning vector first and then cloned into a lentiviral vector (FIG. 1-10). The sequence integrity was confirmed by sequencing. For preparing lentiviral particles, the 293 T cell line was obtained from NCCS Pune and used for transfection. The 293 T cells were cultured in RPMI medium containing 10% fetal bovine serum and L-glutamine without antibiotics. The lentiviral vector was purified using the Maxiprep plasmids extraction kit (Thermofisher, USA) as its concentration was determined using a spectrophotometer (Labman, India). Similarly, the packaging vectors were also purified and concentration was determined. The purified lenti-CD19 CAR vector along with the packaging vectors were transfected into 293T cells. The third generation replication incompetent lentiviral vector along with the packaging vectors was used for this study to ensure lentiviral particles are completely replication incompetent. One the day prior to the transfection, the 293T cells were seeded on to a T25 culture flask in 5 ml of RPMI with 10% FBS without antibiotics. Next day, the flask had a monolayer confluence of 60-70% and it was used for transfection. The plasmids were diluted in 0.5 ml of serum free Opti-MEM medium and the transfection reagent was diluted in another 0.5 ml of Opti-MEM medium. They were allowed to stand for 30 minutes at room temperature. Then, both the plasmid and transfection reagent content were mixed together and allowed to stand for 45 minutes. Meanwhile, the 293T cell flask was taken out from the incubator and the old media was removed and discarded. The monolayer was washed with 2 ml of Opti-MEM medium (ThermoFisher, USA) gently and fresh 1 ml of Opti-MEM was added. Then the plasmid-transfection reagent complex was added to the monolayer gently and mixed well for uniform distribution of the complex. The flask was incubated at 37° C. incubator. After 8 hours of incubation, 2 ml of fresh Opti-MEM medium was added and the flask was kept back in to the CO2 incubator for overnight incubation. Next day, 4 ml of RPMI medium containing 20% FBS was added to the flask and incubated back in to the CO2 incubator. The cells were observed everyday under the inverted microscope as represented in FIG. 11. After 72 hours following transfection, the supernatant containing viral particles was harvested and the concentration of viral particles was determined by measuring the p24 protein using standard commercially available kit. Then it was aliquoted and stored at minus 80° C. in a freezer until further use. These lenti-CD19 CAR viral particles were used to transduce primary human activated T cells to generate CD19 targeting CAR T cells. To further improve the concentration of lentiviral particles generated in the cultures, two transfection reagents were tested using the lentiviral vectors containing red and green fluorescent different reporter gene and found that both were useful for high level expression of genes upon transfection into 293 T cells, however, the transfection reagent SN01 was slightly better than the SN02 as represented in FIG. 12. Transfection of the 293 T cells with three different lentiviral clones carrying anti-CD19 scFV and stained with anti-Fab antibody conjugated with FITC, the stained cells were examined under fluorescent microscope which showed the Clone 13287 and 8T021 were highly expressing the scFV than the clone 1526 as represented in FIG. 13. The lenti-19 CAR viral 8T0121 vector was used for transfecting the 293T cells and the expression of CAR molecules was confirmed by flow cytometer. The transfected 293 T cells were harvested gently through enzymatic treatment and then stained with anti-Fab-FITC antibody and analysed in a flow cytometer (ThermoFisher, USA) The flow analysis confirmed the expression of CAR on the transfected 293T cells as represented in FIG. 14.

### EXAMPLE 2: ACTIVATION AND EXPANSION OF THE T CELLS

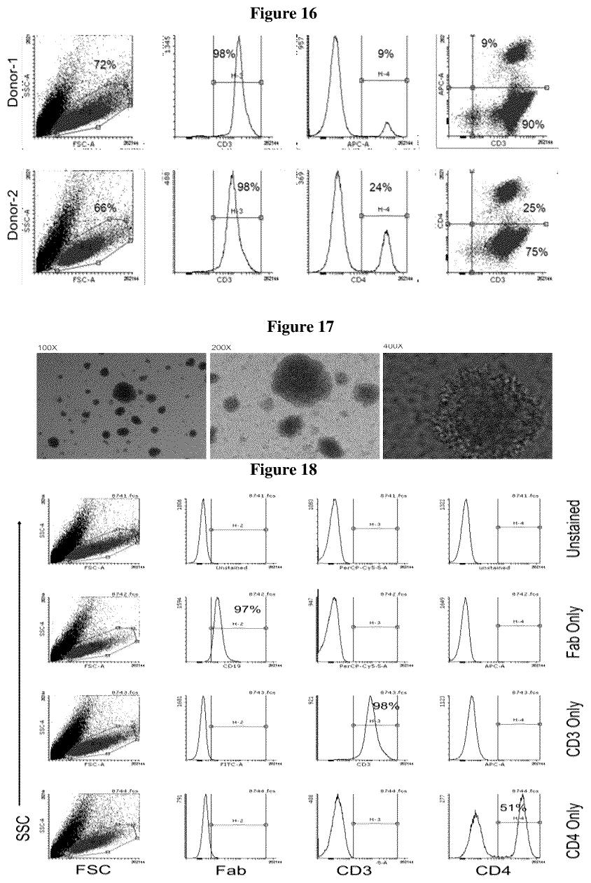

Peripheral blood T cells obtained from normal human donor were activated using soluble anti-CD3 and anti-CD28 antibodies (2.5 μl/ml each, Invitrogen, USA) in the presence of IL2 at the concentration of 100 IU/ml. The morphology and proliferating clumps of cells has been represented in the FIG. 15. Activated T Cells were cultured in the CO2 incubator by providing fresh medium once in every three days after replacing the old medium for the optimal proliferation. The activated and proliferating T cells were stained with labelled antibodies as mentioned below and analysed in a flow cytometer to determine the purity of activated and expanded T cells. The activated and expanded T cells were stained by incubating them with the flurochrome labelled antibodies such as anti-CD3-PerCP and anti-CD4-APC for 20 minutes in dark. The stained cells were washed twice using phosphate buffered saline containing 1% fetal bovine serum and 0.09% sodium azide. The cells were pelleted following centrifugation at 2700 rpm for 4 minutes. The cell pellet was suspended in 1% paraformaldehyde. The stained and fixed cells were acquired in a flow cytometer. The data was analysed to determine the percentage of CD3+ T cells in the total population of cell to assess the purity of activated and expanded T cells. The percentage of CD4 T cells within CD3+ T cells were also determined as represented in FIG. 16.

### EXAMPLE 3: TRANSDUCTION OF ACTIVATED T CELLS WITH LENTI-CD19 CAR VIRAL PARTICLES

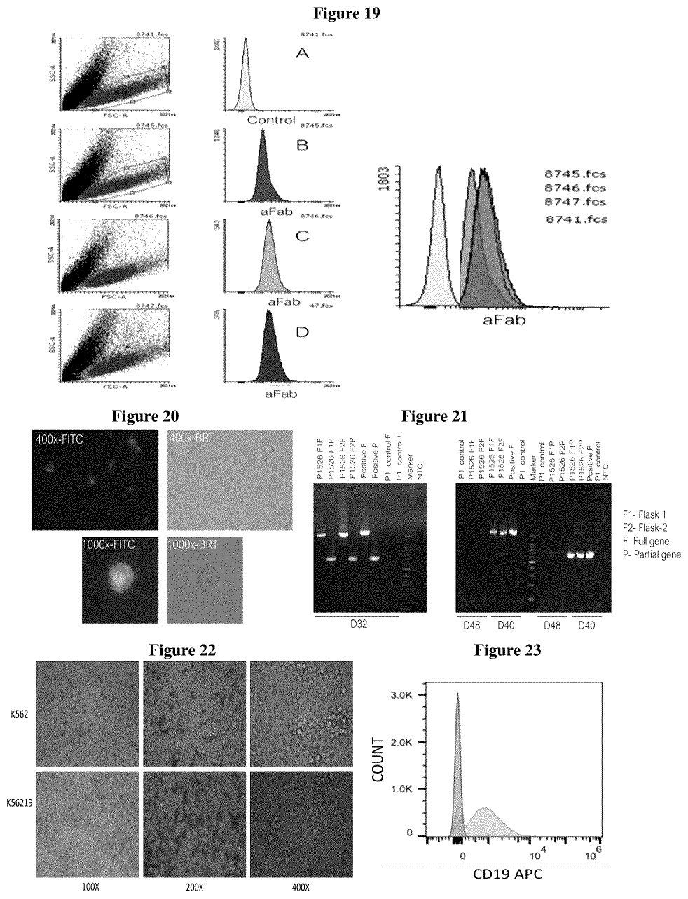

For transducing the T cells with lentiviral particles, the five million peripheral blood mononuclear cells were first activated as described earlier. The soluble anti-CD3 and anti-CD28 mediated activation and robust expansion of T cells was achieved with an increase in the size and number of proliferating clump of T cells in the culture (FIG. 17). The activated cells were characterized by staining them using anti-CD3-PerCP, anti-CD4-APC, and anti-Fab-FITC and analysing them in a flow cytometer. The lenti-19 CAR viral particles were used for transducing the proliferating T cells by incubating them together at 37° C. for overnight, 12-16 h. They were cultured for few weeks following transduction by providing them with fresh medium containing IL2 once in every 2-3 days. During the culture, an aliquot of cells was taken and stained with anti-Fab-FITC antibody (Jackson immunoresearch, USA) and analysed in a flow cytometer (Beckman Coulter, USA). The cells were also stained with specific individual and combination of antibodies to detect the CD3 (ThermoFisher, USA), CD4 (BD Bioscience, USA) and CAR binding anti-Fab antibody. The stained cells were analysed for CAR expression using a flow cytometer as represented in FIG. 18 and FIG. 19. The specific staining of each antibody was observed without any spectral overlap or nonspecific signal in individual as well as combination tubes. The transduced T cells were stained with FITC labelled CAR binding antibody and the expression of CAR was also observed by analysing the cells under fluorescent microscope as represented in FIG. 20. During the culture, an aliquot of cells was taken and DNA was extracted and tested using specific primer for the presence of traduced CAR transgene and it was detectable up to day 40 as represented in FIG. 21.

### EXAMPLE-4: GENERATION OF K562 CELL EXPRESSING CD19 MOLECULE ON THE SURFACE

K562 cells were procured from NCCS, Pune and cultured in the RPMI medium with 10% foetal bovine serum (ThermoFisher, USA). They were transfected with mammalian expression vector containing CD19 gene (SinoBiological) and selected in the presence of antibiotic Hygromycin (SigmalAldrich) in the culture medium at a concentration of 100 μg/ml. The K562 cells expressing the CD19 gene was selected in the culture in the presence of antibiotics over a period of 3 weeks and the selected cells were expanded by continuing the culture. The parental K562 and the engineered K562 to express CD19 (K562-19) were represented in the FIG. 22. The expression of CD19 on the surface of engineered K562 cells was confirmed by staining the cells with anti-CD19-APC antibody and analysing them in the flow cytometer (BecmanCoulter, USA). The expression of CD19 on the surface of K562 cells is represented in the FIG. 23.

### EXAMPLE 5: CYTOTOXICITY OF CD19 SPECIFIC CAR T CELLS ON CD19+K562 CELL LINE DURING CO-CULTURE

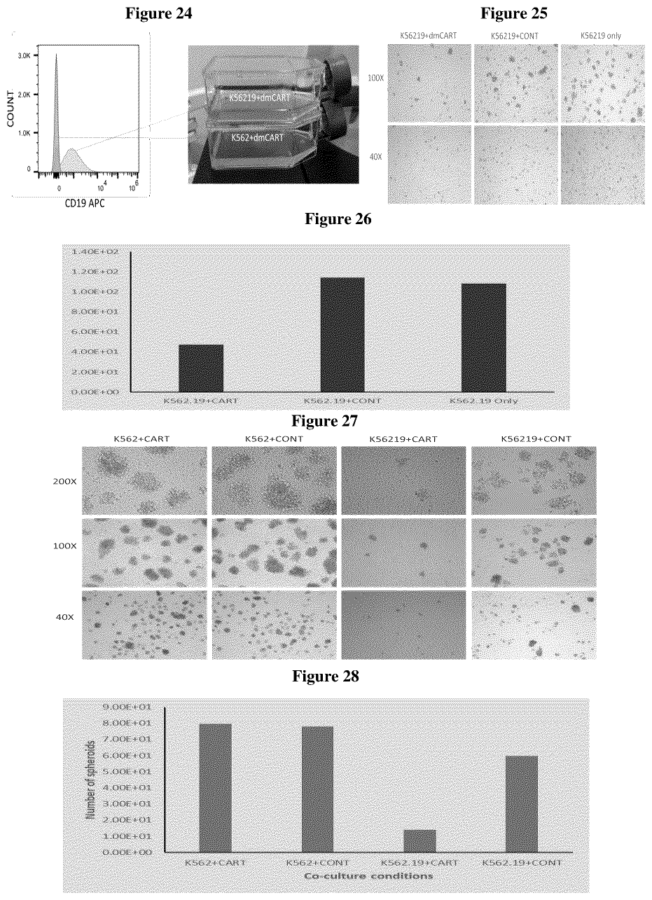

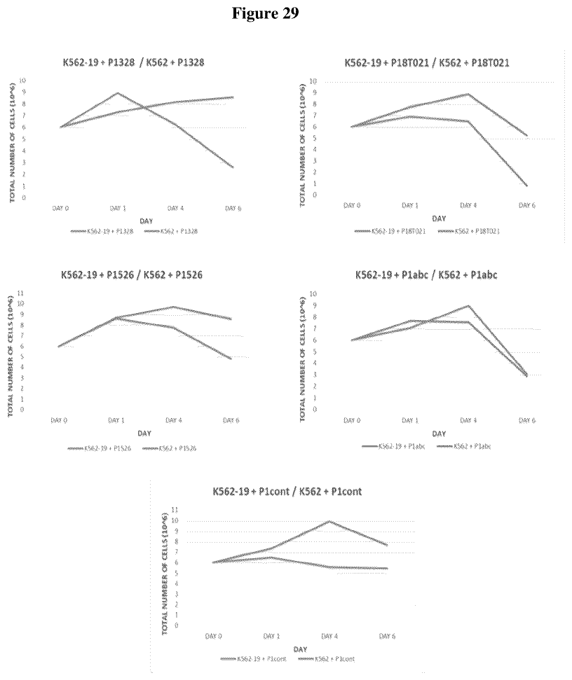

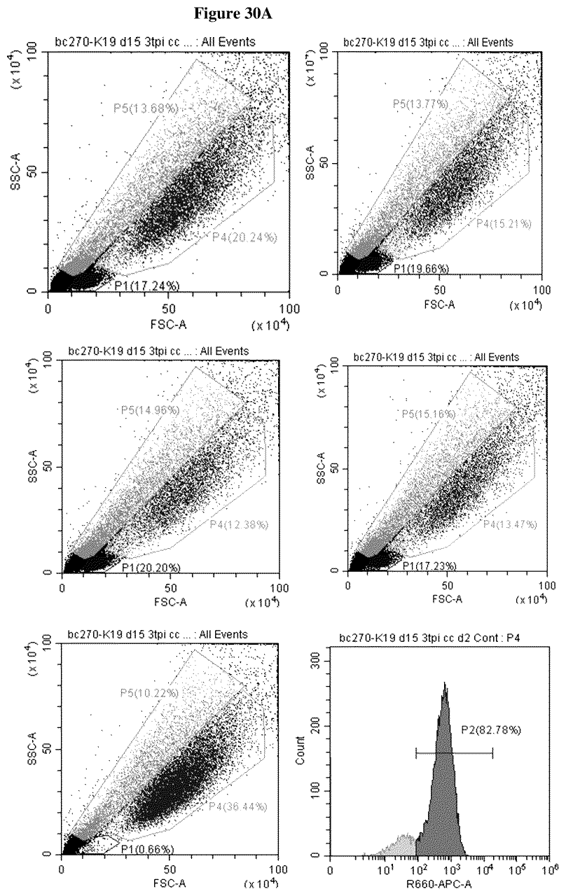

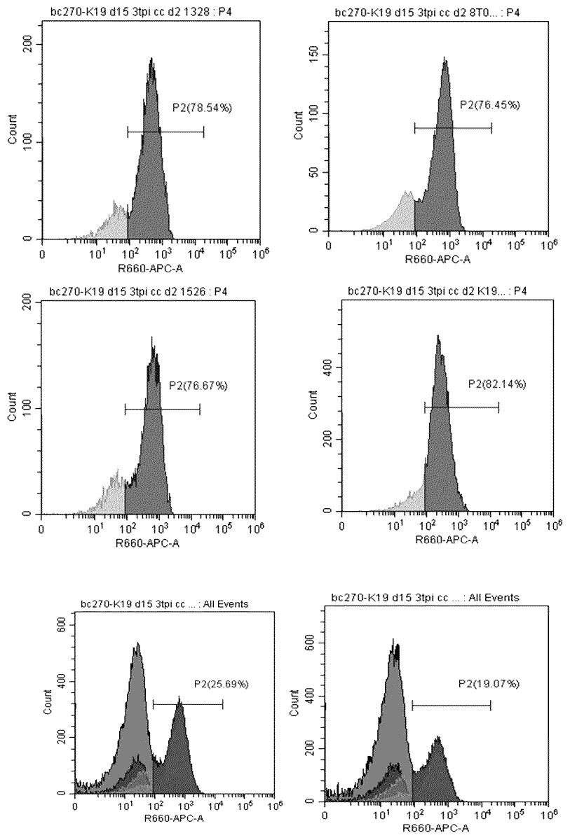

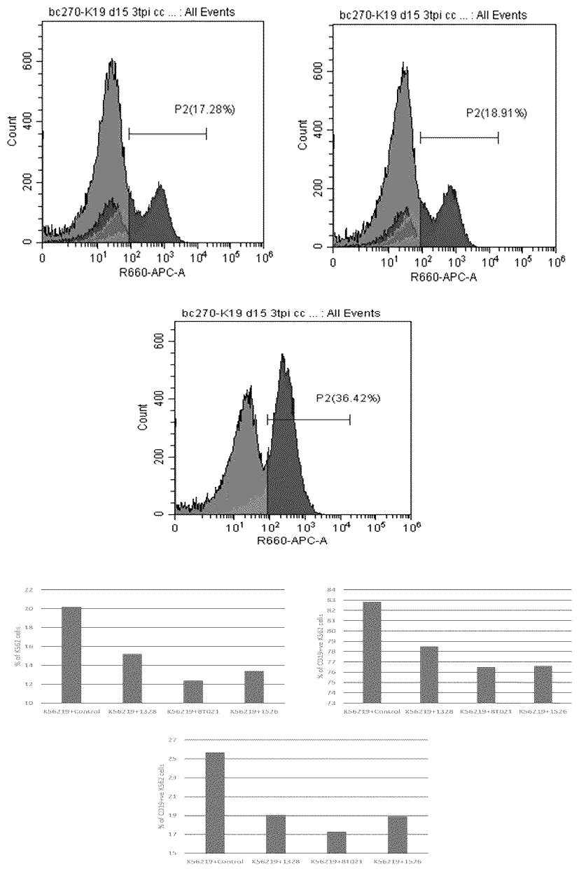

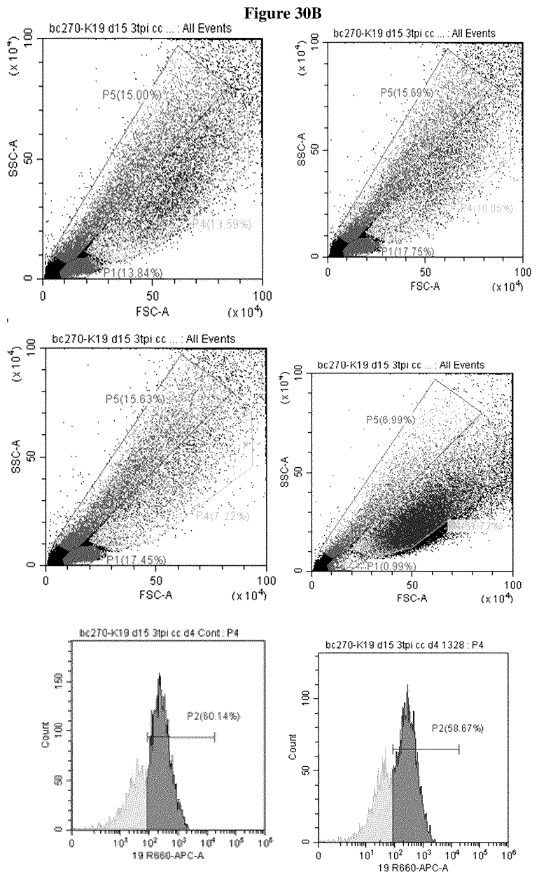

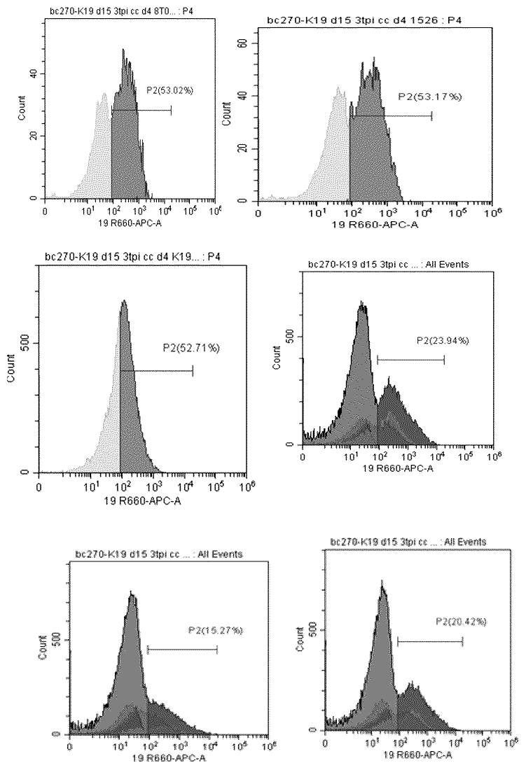

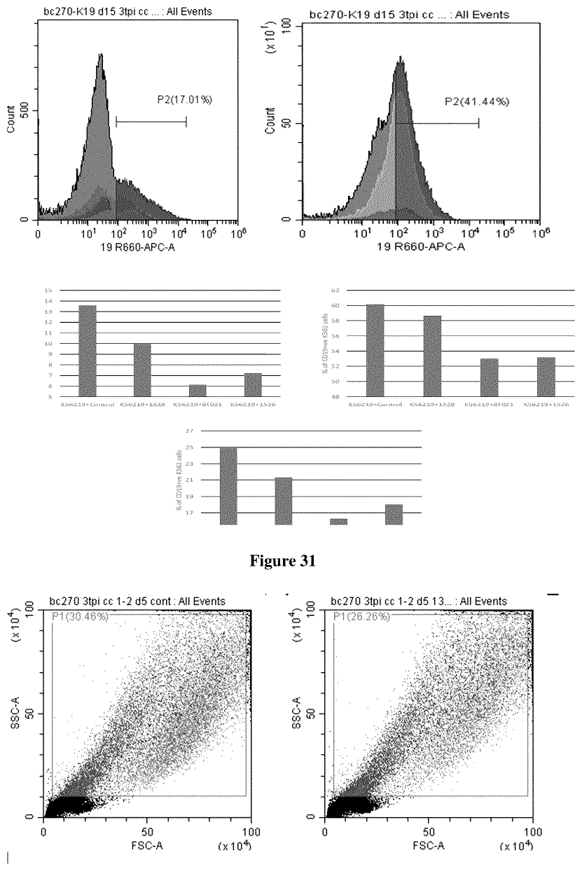

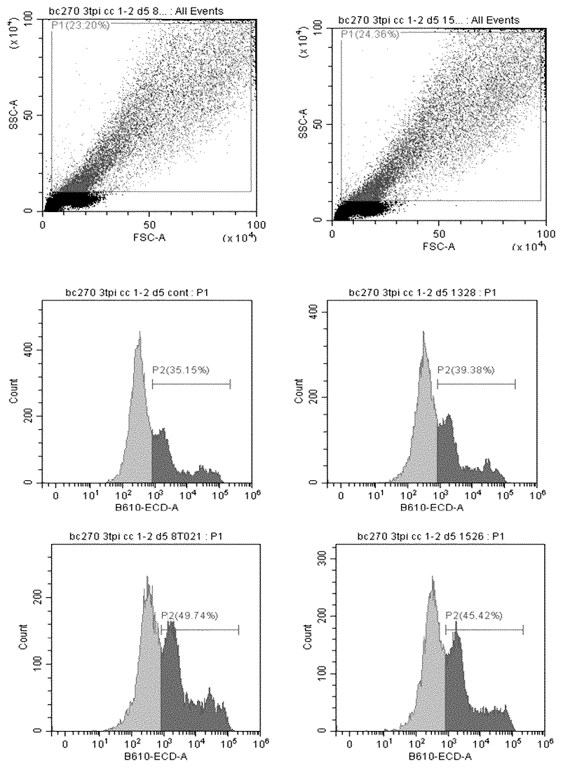

When the CD19 specific CAR T cells were co-cultured with the K562 cells and K562-expressing CD19 (K562-19), it was found that CD19 specific CAR T cells were able to kill the CD19 expressing K562 cells in the specific manner, but not able to kill the normal K562 cells, which is not expressing CD19 as represented in FIG. 24. The Spheroids of the K562 cell were measured in the coculture to quantify the clonal proliferation of K562 cells in the presence of CD19 specific CAR T cells or control T cells without CAR. Initially, coculture experiment was carried out to understand the specific killing of target K562-19 cells only by CD19 specific CAR T cells and not by the control T cells without CAR as represented in FIG. 25, which showed that K562-19 cells were specifically killed by CD19 specific CAR T cells as represented in FIG. 26. Later, the coculture was carried out with two types of K562 cells (with and without CD19 expression), and two types of T cells (with and without CD19 specific CAR expression), which confirmed that the CD19 specific CAR T cells generated in the laboratory are able to specifically kill the CD19 expressing K562 cells as represented in FIGS. 27 and 29. K562-19 and CAR T cells were used at the ratio of 1:2, 1:5, and 1:10 for the coculture and observed the cells for up to 6 days. Analysis of the cells using flow cytometer and determined the total K562 cells and the cells expressing CD19 with and without gating as shown in the FIG. 30. It was able to observe quantitative difference between the control T cells and CAR T cells in terms of killing and elimination of the K562 cells. Stained the cocultured cells with propidium iodide and analysed using the flow cytometer and able to observe increased number of dead cells up taking PI in the CAR T cells than the control T cells cocultured with K562-19 cells as represented in the FIG. 31. Of the 3 different versions of CAR T cells generated, there was some difference in their cytotoxic potential to kill the cancer cells.

### EXAMPLE-6: STUDYING THE DOSE DEPENDENT CYTOTOXICITY OF THE CAR T CELLS ON THE K562-19 CELLS

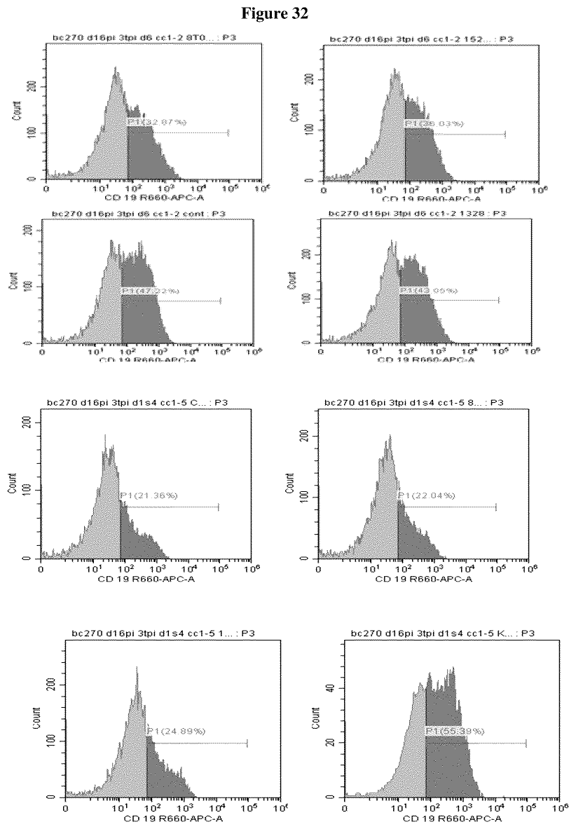

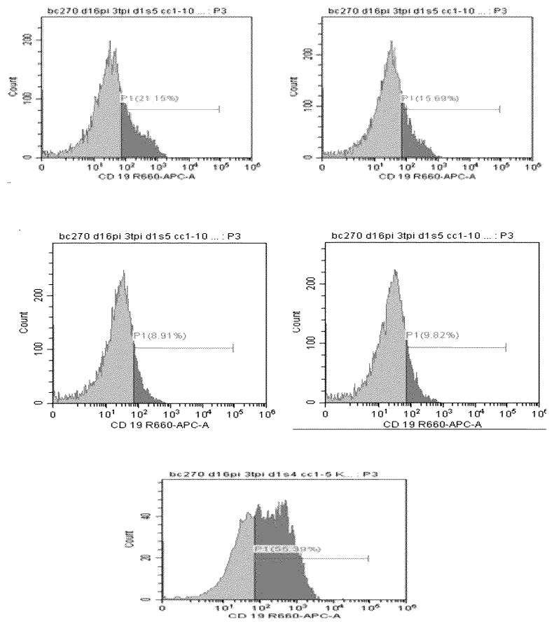

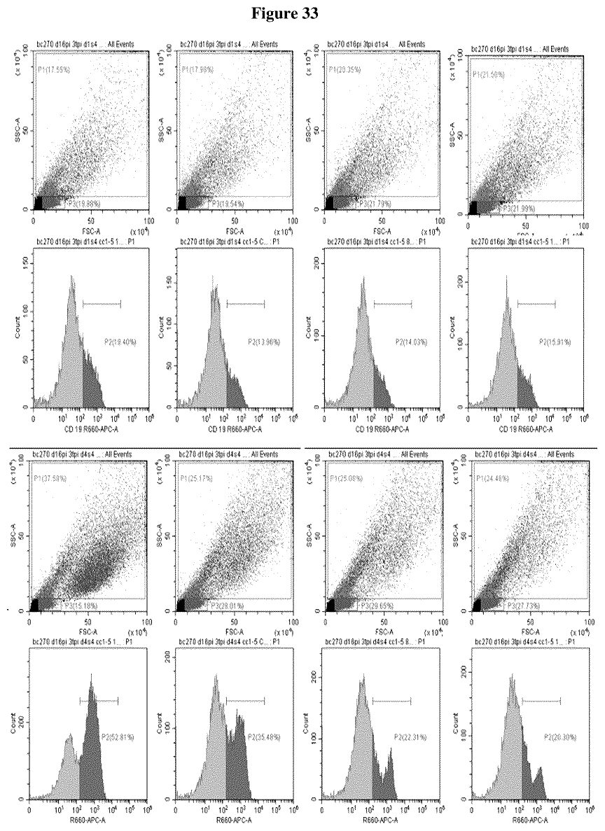

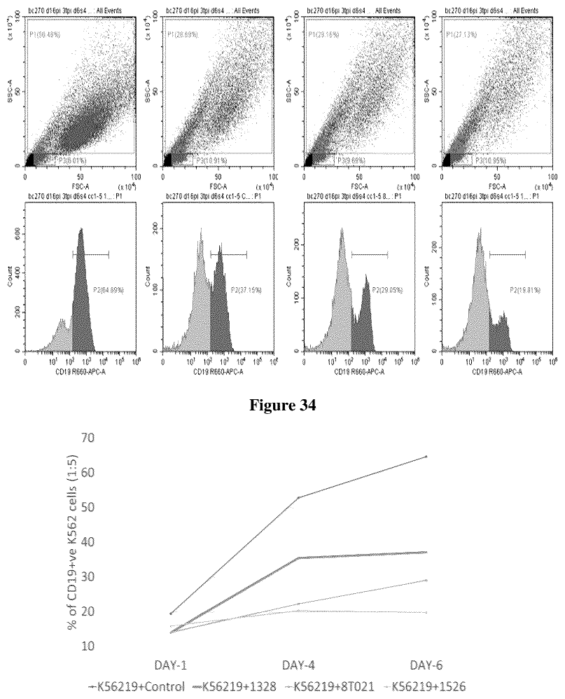

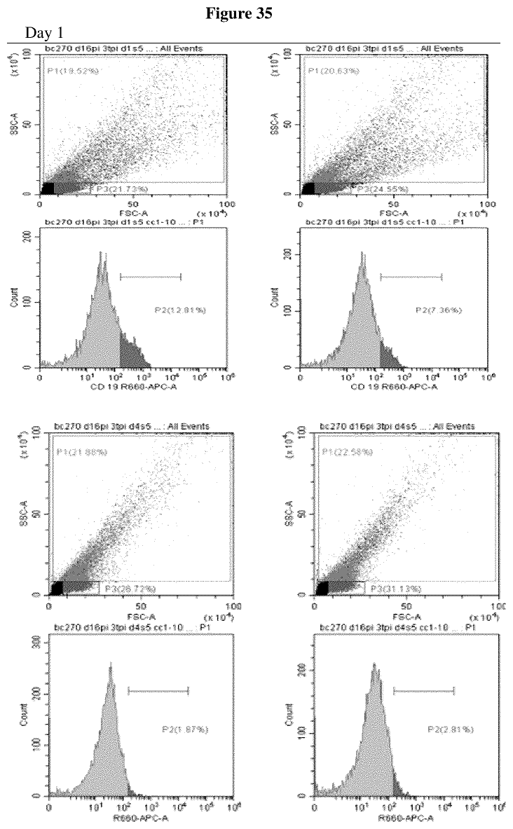

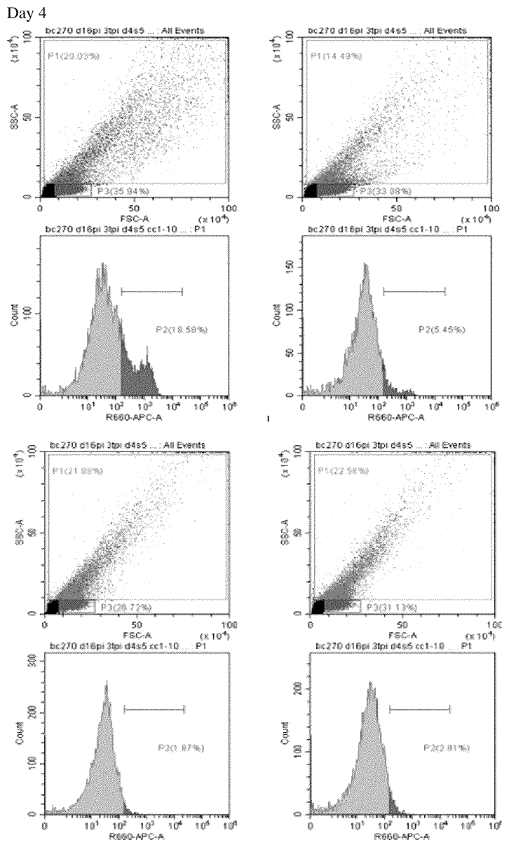

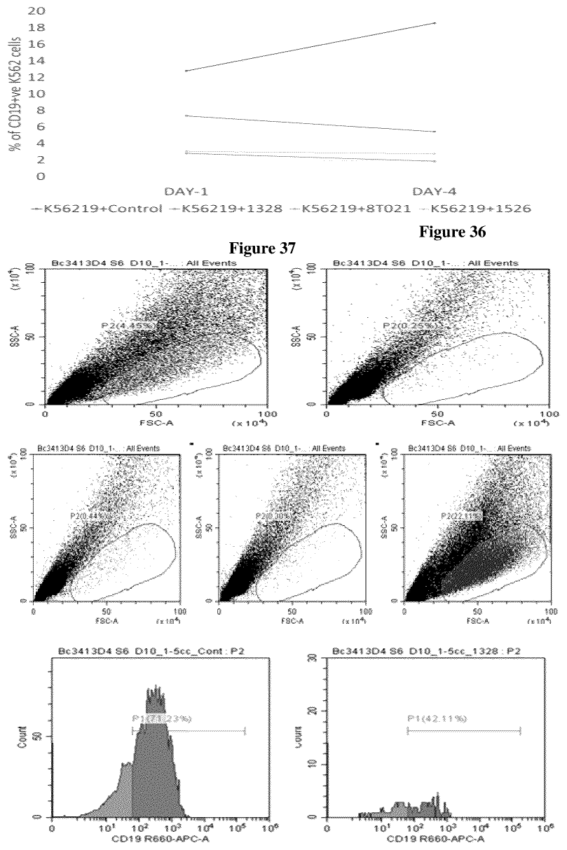

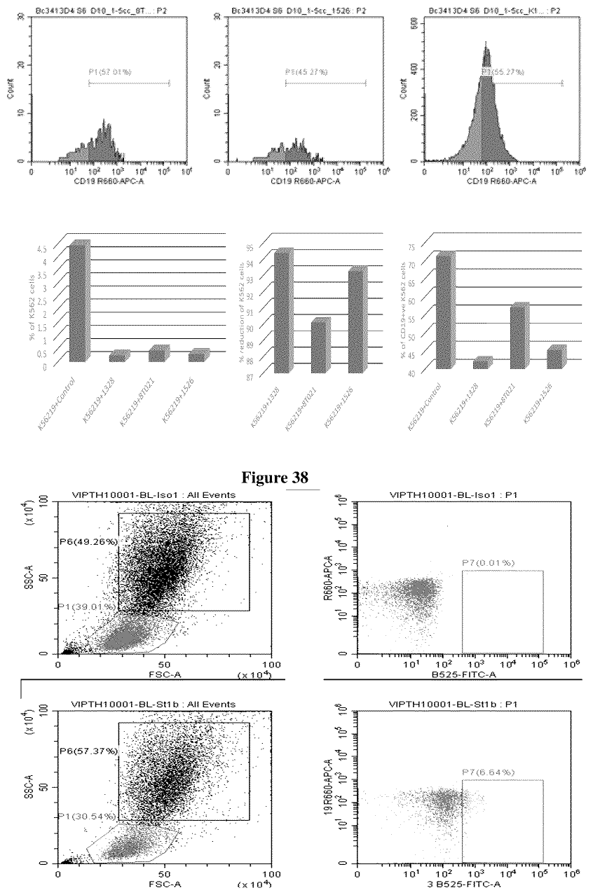

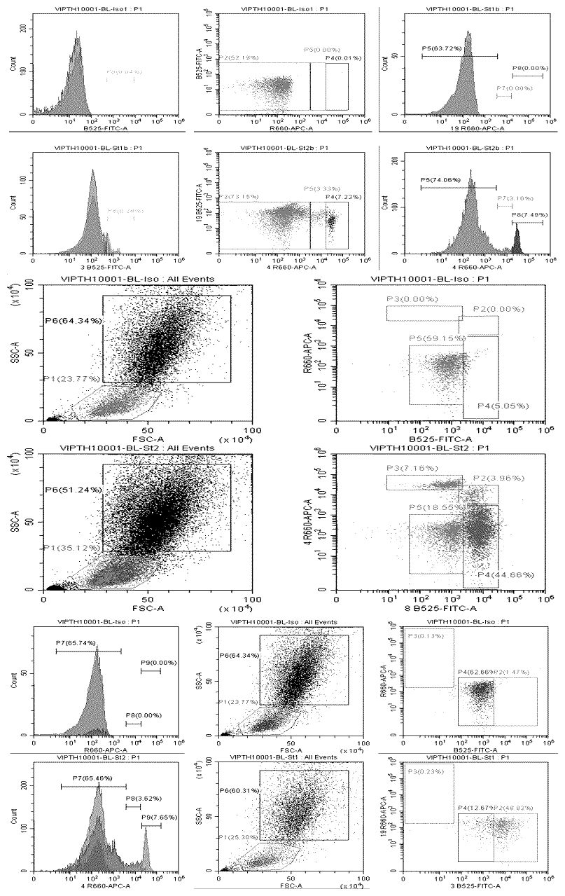

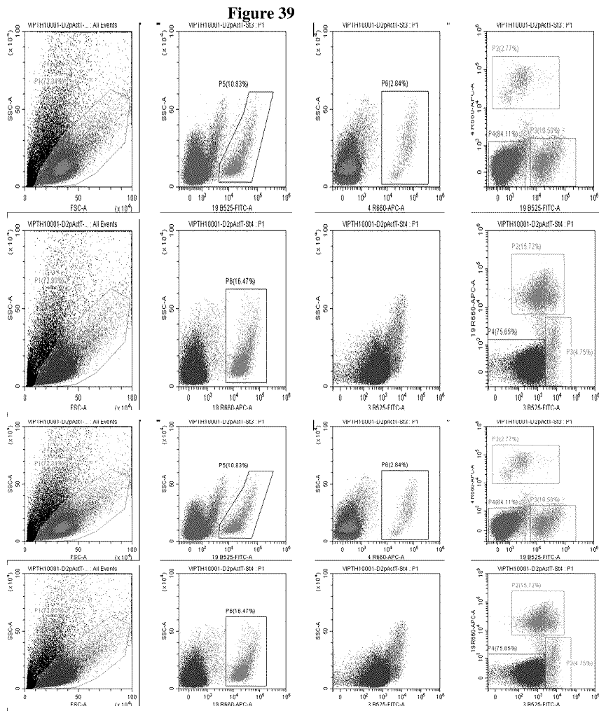

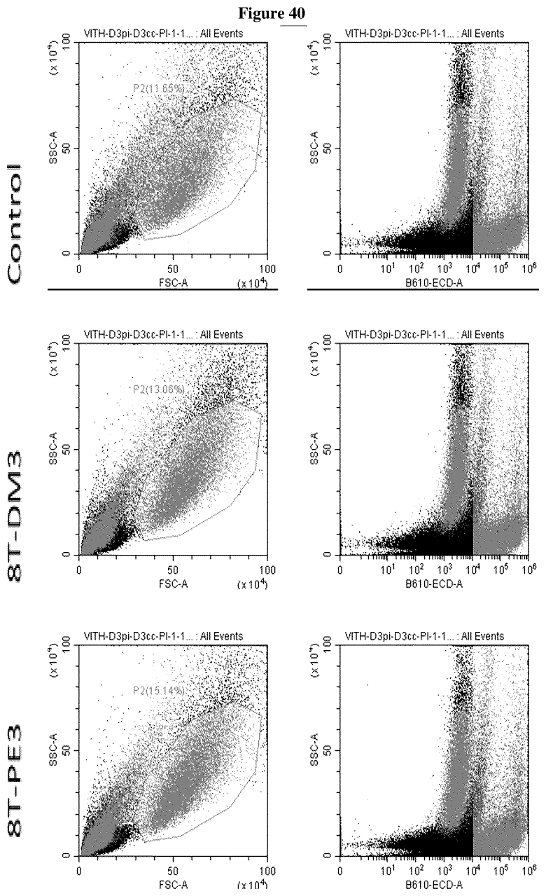

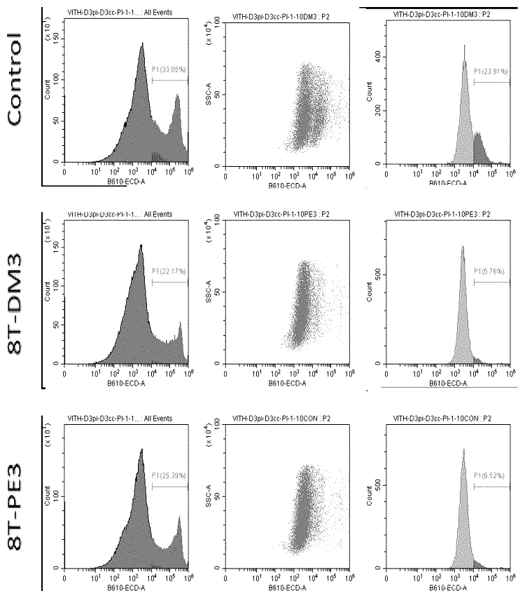

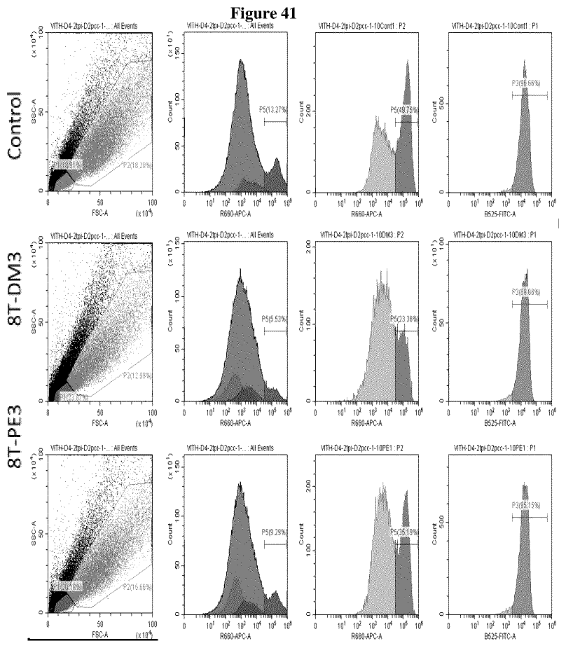

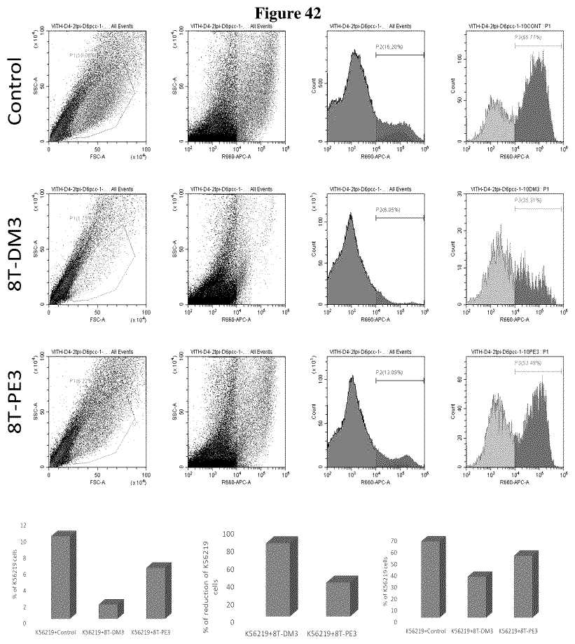

For this, cancer cells and T cells at the ratio of 1:2, 1:5, and 1:10 were used. The cells on different days were analysed by flow cytometry and quantified the K562-19 cells and compared the control T cells and CAR T cells for their cytotoxic potential. The result is represented in FIG. 32. Also studied the dose dependent effect over the period of time following the cocultured cells for up to day 6 at the ratio of 1:5 and 1:10 and the results are shown in FIGS. 33 and 34. As the image shows the first column in the left is cancer cells cocultured with control T cells whereas the other columns are cancer cells cocultured with CAR T cells. The cancer cells number kept increasing in the presence of control T cells and they decreased in the presence of CAR T cells. The FIGS. 33 and 34 indicates the 1:5 ratio of coculture and the FIGS. 35 and 36 indicates the 1:10 ratio of coculture. FIGS. 34 and 36 shows the change in cancer cell number over period of time. Complete elimination of cancer cells on the coculture at 1:5 ratio was achieved as represented in FIG. 37. This study showed that the CAR T cells that were generated in the laboratory using the protocol have the functional ability to kill the cancer cells in a specific manner. They can specifically recognize the CD19 molecule on the cancer cells and able to kill them. The experiments to understand the functional activity of the CAR T cells generated from patient suffering from relapsed B-ALL for the specific killing the K562 cells expression CD19 on the surface was done. The patient blood was collected and stained the cells with antibodies and analysed using a flow cytometer to determine the presence of T cells as represented in FIG. 38. Then, T cells were obtained and expanded them on culture and characterized by flow cytometry as represented in FIG. 39. The T cells were converted in to CD19 specific CAR T cells by transducing with the lentiviral particles. The efficacy of patient derived CD19 specific CAR T cells for the killing of CD19 expressing K562 cells was studied. The K562-19 cells were cocultured with the CAR T cells and studied its CD19 specific killing target cells as represented in FIGS. 40 and 42. K562-19 and CAR T cells were used at the ratio of 1:2, 1:5, and 1:10 for the coculture and observed the cells for up to 6 days. Analysis of the cell by staining with propidium iodide was done as represented in FIG. 41. Also analysed the cells using flow cytometer and determined the total K562 cells with and without gating as represented in the FIG. 42. Quantitative difference between the control T cells and CAR T cells was observed in terms of killing and elimination of the K562 cells.

1. A Chimeric antigen receptor (CAR) T cell product comprising third generation chimeric antigen receptor gene CD19-CD28-41BB-CD3z comprising the nucleotide sequences of single chain variable region of anti-CD19 antibody, signalling domain of CD28, signalling domain of 41BB, transmembrane region CD8a, CD8aHinge region, and CD3z signalling domain along with leader and linker sequences, where in the CAR gene nucleotide sequence is as per SEQ ID No. 1, which is further used by cutting to remove the 41BB domain to generate the second generation CD19-CD28-CD3z CAR gene as per SEQ ID No. 2 and cutting to remove the CD28 domain to generate the CD19-41BB-CD3z CAR gene as per SEQ ID No. 3 to generate CAR T cell products.

2. (canceled).

3. (canceled).

4. The CAR T cell products as claimed in claim 1, wherein the antibody portion or the signalling domains can be switched by restriction digestion using restriction enzymes.

5. The CAR T cell products as claimed in claim 1, wherein the CAR gene sequence comprising DNA encoding a chimeric antigen receptor (CAR) protein which is at least 90% identical to SEQ ID No: 1, 2, and 3.

6. (canceled).

7. The CAR T cell products as claimed in claim 1, wherein the CAR T cell products are created by polymerase chain reaction and restriction enzyme which cuts at the critical junction without compromising the functional integrity of CAR gene.

8. The CAR T cell products as claimed in claim 1, wherein restriction enzymes is selected from Hpa 1, which is inserted between the hinge region and single chain antibody sequence, and Bam H1, which is located at the junction between CD3z signalling domain sequence and co-stimulatory domain sequence, use of combinations of these restriction enzymes or along with suitable restriction enzymes to cut the sequences in upstream or downstream to replace the single chain variable chain of antibody sequence or signalling domain sequences to generate new CAR T cell product.

9. The CAR T cell products as claimed in claim 1, wherein the CAR T cell product allows replacing the antibody domain or the signalling domain with an alternative antibody domain or alternative signalling domain without affecting the functional integrity by restriction digestion.

10. The CAR T cell products as claimed in claim 1, wherein the alternative antibody domain is selected from anti-B7-H3, BCMA, CAIX, CD123, CD133, CD138, CD147, CD19, CD22, CD30, CD33, CD38, CD4, CD5, CD7, CD70, CEA, EGFR, EGFRVIII, FAP, EpCAM, FOLR1, GPC3, Her2, Her3, HGFR, IL13RA2, MSLN, MUCI, MUC16, Nectin-4, NKG2D, PSCA, PSMA, ROR1, SLAMF7, uPAR and VGFR2 antibody sequences.

11. The CAR T cell products as claimed in claim 1, wherein the alternative signaling domain are selected from CD28, 41BB, ICOS, OX40 and FcεR1-γ.

12. (canceled).

13. The CAR T cell products as claimed in claim 1, wherein the CAR T cell product is used for parenteral or local administration in the patient.

14. The CAR T cell products as claimed in claim 1, wherein the CAR gene sequence is cloned into a DNA cloning vector, expression vector, lentiviral vector, adenoviral vector, adeno-associated viral vector, and transposon vector.

15. The CAR T cell products as claimed in claim 1, wherein the CAR T cell product is an engineered cell comprising DNA encoding a chimeric antigen receptor (CAR) protein comprises an amino acid sequence which is at least 90% identical to SEQ ID No: 4 or 5 or 6.

16. The CAR T cell products as claimed in claim 1, wherein DNA encoding the CAR is integrated into the genome of the cell or remain as an extra chromosomal DNA.

17. (canceled).

18. (canceled).

19. (canceled).

20. (canceled).

21. The CAR T cell products as claimed in claim 1, wherein the CAR T cell product is manufactured using the soluble anti-CD3 and anti-CD28 antibodies based activation of T cell without utilizing anti-CD3 bead or anti-CD28 bead.

22. The CAR T cell products as claimed in claim 1, wherein the CAR T cell product is manufactured using T cells of peripheral blood mononuclear cells with or without any purification of T cells, where the T cells are activated using anti-CD3 and anti-CD28 antibodies and addition of interleukin, wherein said interleukin are selected from interleukin-2, interleukin-15, interleukin-12, interleukin-18, interleukin-21 and interleukin-7, and then infecting using CAR gene containing viral particles to generate CAR T cells.

23. The CAR T cell products as claimed in claim 1, wherein the CAR T cell product is manufactured using T cells recovered from the in vitro cultures of blood mononuclear cells, macrophages, nurse macrophages, and lympho-myeloid niches following activation and/or addition of interleukin and infecting them with CAR gene containing viral particles to generate CAR T cells, wherein said interleukin are selected from interleukin-2, interleukin-15, interleukin-12, interleukin-18, interleukin-21 and interleukin-7.

24. (canceled).

25. The CAR T cell products as claimed in claim 1, wherein the CAR gene sequence containing vectors are used in the process of transfecting 293T cells for generating viral particles containing CAR gene, which are then used for the generation of CAR T cell products.
